(19) United States
(12) Patent Application Publication
(10) Pub. No.: US 2026/0234680 A1
(43) Pub. Date: Aug. 13, 2026

(54) XANTHOMONAS CAMPESTRIS STRAIN WITH HIGH PRODUCTION OF TEMPERATURE-RESISTANT AND RAPID-DISSOLVING XANTHAN GUM AND USE THEREOF

(71) Applicant: INNER MONGOLIA UNIVERSITY OF TECHNOLOGY, Huhhot (CN)

(72) Inventors: Zhanying LIU, Huhhot (CN); Han SUN, Huhhot (CN)

(73) Assignee: INNER MONGOLIA UNIVERSITY OF TECHNOLOGY, Huhhot (CN)

(21) Appl. No.: 19/151,636

(22) PCT Filed: Dec. 17, 2024
(86) PCT No.: PCT/CN2024/139785

§ 371 Date: Jul. 29, 2025

(30) Foreign Application Priority Data
Dec. 21, 2023 (CN) 202311763508.2

(51) Int. Cl. C12P 19/06

(52) U.S. Cl. CPC C12P 19/06

(57) ABSTRACT

A Xanthomonas campestris strain with high production of temperature-resistant and rapid-dissolving xanthan gum and use thereof are provided. Xanthomonas campestris Y-11 has a deposit number of GDMCC No: 63460. The Xanthomonas campestris strain Y-11 has a xanthan gum production rate of 10-20% higher than that of an original strain, and the xanthan gum obtained by fermentation has properties such as rapid dissolution, agglomeration resistance, and high-temperature tolerance, making it suitable for the fermentative production of temperature-resistant and rapid-dissolving xanthan gum. Meanwhile, genetic stability analysis is performed on the Xanthomonas campestris strain. The strain is subjected to continuous passage culture, and determined for xanthan gum production rates every two passages. The xanthan gum production rate and gum quality do not significantly change after 12 consecutive passages, demonstrating good stability of the strain.

### CROSS REFERENCE TO THE RELATED APPLICATIONS

This application is the national phase entry of International Application No. PCT/CN2024/139785, filed on Dec. 17, 2024, which is based upon and claims priority to Chinese Patent Application No. 202311763508.2, filed on Dec. 21, 2023, the entire contents of which are incorporated herein by reference.

### TECHNICAL FIELD

The present application belongs to the field of microbiological technology and particularly relates to a Xanthomonas campestris strain with high production of temperature-resistant and rapid-dissolving xanthan gum and use thereof.

### BACKGROUND

Xanthan gum, also known as xanthan or hansen gum, is a water-soluble microbial exopolysaccharide produced by the aerobic fermentation of Xanthomonas campestris using carbohydrates as the main raw material. It is currently the most extensively produced microbial polysaccharide globally. Xanthan gum is synthesized by Xanthomonas via aerobic fermentation of saccharides, where the 1,6-glycosidic bonds are cleaved, the branched chains are opened and linear chains are formed through 1,4-linkages, resulting in an acidic extracellular heteropolysaccharide. It was discovered in 1963 at the Northern Regional Research Center of the United States Department of Agriculture as polysaccharide B-1459 synthesized by Xanthomonas campestris NRRL B-1459, and was commercially produced in large scale in early 1964. Due to its macromolecular special structure and colloidal properties, xanthan gum has excellent physicochemical properties such as suspensibility, emulsibility, thickenability, pseudoplasticity, thermal stability, etc.. It is widely used in various fields as a thickener, emulsifier, stabilizer, gelling agent, wetting agent, film-forming agent, etc., making it one of the most superior biogums available. Xanthan gum has general properties of long-chain polymers, but it contains more functional groups than general polymers, leading to unique performance under specific conditions. The number of pyruvate groups at the end of molecular side chains of xanthan gum has a significant impact on its performance. In industrial production, aerobic fermentation is performed using Xanthomonas campestris, and processes such as precipitation, centrifugation, washing, separation, drying, grinding and packaging are performed on the fermentation broth to yield the final xanthan gum product.

The selection of strains is a critical issue in fermentation production. Currently, Xanthomonas strains are commonly used in industrial production for the fermentative preparation of xanthan gum. Through fermentation with the strains, the product is secreted extracellularly, and the fermentation broth is processed and extracted to obtain the target product. However, the continuous increase in the number of strain passages leads to degeneration of the existing strains, which directly impacts the industrial production of xanthan gum. Moreover, the resulting xanthan gum products exhibit limited performance, with unsatisfactory high-temperature resistance and solubility, failing to meet the requirements of certain application scenarios. Therefore, in the xanthan gum industry, the search for better strains is very important for further increasing the gum yield and improving the performance of xanthan gum obtained by fermentation to expand the application scenarios.

### SUMMARY

An objective of the present disclosure is to provide a Xanthomonas campestris strain with high production of temperature-resistant and rapid-dissolving xanthan gum and use thereof. This aims to solve the problems of the low gum production rate in the existing xanthan gum industry and the unsatisfactory high-temperature resistance and solubility of the produced xanthan gum.

In order to achieve the objective described above, the present disclosure provides the following technical solutions: The present disclosure provides a Xanthomonas campestris strain with high production of temperature-resistant and rapid-dissolving xanthan gum, where the Xanthomonas campestris strain is designated as Xanthomonas campestris Y-11, has been classified under the taxonomic name of Xanthomonas campestris, and was deposited with the Guangdong Microbial Culture Collection Center on May 12, 2023, under a deposit number of GDMCC No: 63460. The deposit address is 5th Floor, Building 59, 100 Xianlie Middle Road, Guangzhou.

In some embodiments, the screening process for the strain is as follows:
(1) a preserved bacterial solution of an original strain (deposit number: CICC No: 10258) is thawed, and then the preserved bacterial solution is picked up and streaked on a plate medium with an inoculation loop; after formation of single colonies, the single colonies are picked and inoculated at a 1% inoculum size into a liquid seed medium, and then cultured in a thermostatic shaker at 30° C. and 180 r/min for 24 h; two passages are performed to restore the original activity of the original strain;
(2) the activated original strain is inoculated at a 1% inoculum size into a 250 mL Erlenmeyer flask containing 100 mL of liquid seed medium, and then cultured in a thermostatic shaker at 30° C. and 180 r/min for 24 h to obtain liquid seeds at the logarithmic growth phase, such that liquid seed culture is completed;
(3) a bacterial solution of the original strain at the logarithmic growth phase is taken and mixed into a uniform bacterial suspension using a vortex mixer; 1 mL of the prepared bacterial suspension is taken and placed in a 35 mm irradiation dish, sealed with a sealing film, and subjected to irradiation mutagenesis using a 12C6+ ion beam, the ion beam having an extraction energy of 80 MeV/u and a LET of 35.5 keV/mm; a total of 11 mutagenesis doses are selected for irradiation: 0 Gy, 20 Gy, 40 Gy, 60 Gy, 80 Gy, 100 Gy, 120 Gy, 140 Gy, 160 Gy, 180 Gy, and 200 Gy;
(4) the morphology and size of colonies at different irradiation doses are observed, and larger, smoother and more translucent colonies are picked and spot-inoculated onto a screening medium, numbered, and then cultured at 37° C. for 72 h; after formation of colonies, Lugol's iodine solution is added dropwise to the screening medium, and clear zones around the colonies are observed; a colony diameter (C) and a clear zone diameter (H) are measured, and an H/C value is calculated; strains with higher H/C values are inoculated into a liquid medium and cultured in a theramostatic shaker at 30° C. and 180 r/min for 72 h, these strains being taken as primarily screened strains;
(5) the primarily selected strains are numbered, cultured to the logarithmic growth phase, inoculated at a 10% inoculum size into 75 mL of fermentation medium, and then cultured in a thermostatic shaker at 37° C. and 180 r/min for 96 h; after the fermentation is completed, gum production rates and light transmittances of aqueous solutions of xanthan gum fermentation products of the strains are determined; the gum production rates and the aqueous solution light transmittances of the primarily selected strains are compared; optimal strains are selected as secondarily screened strains, and a genetic stability test is performed on the secondarily screened strains; finally, a strain with the highest gum production rate, best aqueous solution light transmittance and optimal genetic stability test results is selected as a target strain.

The present disclosure further provides use of the above-mentioned Xanthomonas campestris strain as a fermentation strain in fermentative preparation of xanthan gum.

In some embodiments, the use includes the following steps:
(1) strain activation: a preserved bacterial solution containing a Xanthomonas campestris strain Y-11 under a deposit number of GDMCC No: 63460 is thawed, and then the preserved bacterial solution is picked up and streaked on a plate medium with an inoculating loop; after formation of single colonies, the single colonies are picked and inoculated at a 1% inoculum size into a liquid seed medium, and then cultured in a thermostatic shaker at a fermentation temperature of 30° C. and 180 r/min for 24 h; two passages are performed to restore original activity of the Xanthomonas campestris strain Y-11;
(2) liquid seed culture: the activated Xanthomonas campestris strain is inoculated at a 1% inoculum size into a liquid seed medium, and then cultured in a thermostatic shaker at a fermentation temperature of 30° C. and 180 r/min for 24 h, such that liquid seed culture is completed;
(3) fermentation culture: the liquid seeds at the logarithmic growth phase are inoculated at a 5-20^{%} inoculum size into a fermentation medium, and then cultured in a thermostatic shaker at a fermentation temperature of 30-40° C. and 180 r/min for 96 h; and
(4) xanthan gum extraction: xanthan gum is extracted from a fermentation broth by an ethanol precipitation method.

In some embodiments, the plate medium in step (1) includes the following components by weight percentage: 0.5% soluble starch, 1% peptone, 0.3% beef extract, 0.5% sodium chloride and 2% agar, pH of the plate medium is 6.5-7.0, where the plate medium is sterilized at 115° C. for 30 min, and culture on the plate medium is performed for 72 h.

In some embodiments, the liquid seed media in step (1) and step (2) both include the following components by weight percentage: 2.0% soluble starch, 0.5% peptone, 0.3% potassium dihydrogen phosphate and 0.2% sodium chloride, and a pH value of the liquid seed medium is 7.0.

In some embodiments, inoculation in step (3) is performed at a 15% inoculum size.

In some embodiments, the fermentation in step (3) is performed at 40° C.

In some embodiments, the fermentation medium in step (3) includes the following components by weight percentage: 4.5% soluble starch, 0.5% glucose, 1.0% soy protein, 0.1% magnesium sulfate heptahydrate, 0.1% dipotassium hydrogen phosphate and 0.1% potassium dihydrogen phosphate, with a pH value of 7.0.

In some embodiments, extracting the xanthan gum from the fermentation broth by the ethanol precipitation method in step (4) includes: mixing the fermentation broth with absolute ethanol, and centrifuging the mixture to obtain the xanthan gum.

In some embodiments, before mixing with absolute ethanol, the fermentation broth is diluted by mixing with deionized water to obtain a diluted solution, with a volume ratio of the diluted solution to the deionized water being 1:3.

The present disclosure provides a method for fermentative preparation of xanthan gum by a Xanthomonas campestris strain Y-11 as a fermentation strain, the method including the following steps:
(1) strain activation: a preserved bacterial solution containing a Xanthomonas campestris strain under a deposit number of GDMCC No: 63460 is thawed, and then the preserved bacterial solution is picked up and streaked on a plate medium with an inoculating loop; after single colonies appearance, the single colonies are picked and inoculated at a 1% inoculum size into a liquid seed medium, and then cultured in a thermostatic shaker at a fermentation temperature of 30° C. and 180 r/min for 24 h; two passages are performed to restore original activity of GDMCC63460;
(2) liquid seed culture: the activated Xanthomonas campestris strain is inoculated at a 1% inoculum size into a liquid seed medium and then cultured in a thermostatic shaker at a fermentation temperature of 30° C. and 180 r/min for 24 h, such that liquid seed culture is completed;
(3) fermentation culture: the liquid seeds at the logarithmic growth phase are inoculated at a 5-20% inoculum size into a fermentation medium, and then cultured in a thermostatic shaker at a fermentation temperature of 30-40° C. and 180 r/min for 96 h; and
(4) xanthan gum extraction: xanthan gum is extracted from a fermentation broth by an ethanol precipitation method.

In some embodiments, the plate medium in step (1) includes the following components by weight percentage: 0.5% soluble starch, 1% peptone, 0.3% beef extract, 0.5% sodium chloride, and 2% agar, pH of the plate medium is 6.5-7.0, where the plate medium is sterilized at 115° C. for 30 min, and culture on the plate medium is performed for 72 h.

In some embodiments, the liquid seed media in step (1) and step (2) both include the following components by weight percentage: 2.0% soluble starch, 0.5% peptone, 0.3% potassium dihydrogen phosphate, and 0.2% sodium chloride, with a pH value of 7.0.

In some embodiments, inoculation in step (3) is performed at an inoculum size of 15%.

In some embodiments, the fermentation in step (3) is performed at 40° C.

In some embodiments, the fermentation medium in step (3) includes the following components by weight percentage: 4.5% soluble starch, 0.5% glucose, 1.0% soy protein, 0.1% magnesium sulfate heptahydrate, 0.1% dipotassium hydrogen phosphate and 0.1% potassium dihydrogen phosphate, with a pH value of 7.0.

In some embodiments, extracting the xanthan gum from the fermentation broth by the ethanol precipitation method in step (4) includes mixing the fermentation broth with absolute ethanol, and centrifuging the mixture to obtain the xanthan gum.

In some embodiments, before mixing with absolute ethanol, the fermentation broth is diluted by mixing with deionized water to obtain a diluted solution, with a volume ratio of the diluted solution to the deionized water being 1:3.

Compared with conventional technology, embodiments of the present disclosure have the following beneficial effects:

1. The Xanthomonas campestris strain provided in the present disclosure (deposit number: GDMCC No: 63460) has a xanthan gum production rate 10-20% higher than that of the original strain, and the xanthan gum obtained by fermentation has properties such as rapid dissolution, agglomeration resistance, and high-temperature tolerance, making it suitable for the fermentative production of temperature-resistant and rapid-dissolving xanthan gum.

2. In the present disclosure, genetic stability analysis is performed on the Xanthomonas campestris strain under a deposit number of GDMCC No: 63460. The strain is subjected to continuous passage culture, and determined for xanthan gum production rates every two passages. It can be seen that the xanthan gum production rate and gum quality of the Xanthomonas campestris strain exhibited no significant change after 12 consecutive passages, demonstrating its good stability. Therefore, the strain can be used as an industrial production strain for the scale-up production of temperature-resistant rapid-dissolving xanthan gum.

### BRIEF DESCRIPTION OF THE DRAWINGS

To describe the technical solutions in the examples of the present disclosure or in the prior art more clearly, the accompanying drawings required for the examples will be briefly described below.

FIG. 1 is a graph showing the growth curve of the original strain (deposit number: CICC No: 10258) of the present disclosure;

FIG. 2 is a line graph showing the relationship between the lethality rate of the original strain (deposit number: CICC No: 10258) of the present disclosure and the irradiation dose;

FIG. 3 is a line graph showing the relationship between the positive mutation rate of the original strain (deposit number: CICC No: 10258) of the present disclosure and the irradiation dose;

FIG. 4 is an analytical bar graph showing the gum production rates of the primarily screened strains of the present disclosure;

FIG. 5 is an analytical bar graph showing the gum production rates of the secondarily screened strains of the present disclosure;

FIG. 6 is an analytical bar graph showing the genetic stability of gum production rates of the Xanthomonas campestris strain (deposit number: GDMCC No: 63460) and the original strain (deposit number: CICC No: 10258) of the present disclosure;

FIG. 7 is an analytical bar graph showing the complete dissolution time of the xanthan gum fermentation products of the Xanthomonas campestris strain (deposit number: GDMCC No: 63460) and the original strain (deposit number: CICC No: 10258) of the present disclosure at a mass concentration of 1%; and

FIG. 8 is a line graph showing the temperature resistance determination of the xanthan gum fermentation products of the Xanthomonas campestris strain (deposit number: GDMCC No: 63460) and the original strain (deposit number: CICC No: 10258) of the present disclosure.

### Biological Deposition

Xanthomonas campestris Y-11, deposited with the Guangdong Microbial Culture Collection Center on May 12, 2023, under a deposit number of GDMCC No: 63460, with a deposit address: 5th Floor, Building 59, 100 Xianlie Middle Road, Guangzhou.

Original strain Xanthomonas campestris, deposited with China Center of Industrial Culture Collection on Nov. 13, 1998, under a deposit number of CICC No: 10258, with a deposit address: Building 6, No. 24 Jiuxiangiao Middle Road, Chaoyang District, Beijing.

### DETAILED DESCRIPTION OF THE EMBODIMENTS

The present disclosure will be further described in conjunction with various embodiments, but the embodiments of the present disclosure include, but are not limited to, the following examples.

The Xanthomonas campestris strain (deposit number: GDMCC No: 63460) of the present disclosure was obtained by performing radiation mutagenesis on the original strain Xanthomonas campestris (deposit number: CICC No: 10258, hereinafter referred to as “original strain”) by heavy ion beam radiation mutagenesis technology, followed by screening and selection. The mutagenesis technology and screening process were as follows:

### 1. Strain Culture

The bacterial solution of the original strain was thawed. A small amount of the bacterial solution was picked up and streaked on a plate medium with an inoculating loop. After the formation of single colonies, the single colonies were picked and inoculated into a liquid seed medium, and then cultured in a thermostatic shaker at 30° C. and 180 r/min for 24 h. Then, the culture was inoculated at a 1% inoculum size into a 250 mL Erlenmeyer flask containing 100 mL of liquid seed medium, and then cultured in a thermostatic shaker at 30° C. and 180 r/min for 24 h. Two passages were performed to restore the activity of the original strain.

The plate medium composed of 0.5% soluble starch, 1% peptone, 0.3% beef extract, 0.5% sodium chloride and 2% agar, pH 6.5-7.0, was sterilized at 115° C. for 30 min.

### 2. Liquid Seed Culture

The activated original strain was inoculated at a 1% inoculum size into a 250 mL Erlenmeyer flask containing 100 mL of liquid seed medium, and then cultured in a thermostatic shaker at 30° C. and 180 r/min for 24 h, such that liquid seed culture was completed.

The liquid seed medium composed of 2.0% soluble starch, 0.5% peptone, 0.3% potassium dihydrogen phosphate and 0.2% sodium chloride, pH 7.0, was filled at 100 mL/250 mL.

### 3. Growth Curve Determination

The bacterial solution of the original strain after two to three passages was inoculated at a 1% inoculum size into 100 mL of liquid seed medium, and then cultured in a thermostatic shaker at 30° C. and 180 r/min. The absorbance of the seed medium inoculated at 0 h was determined at a wavelength of 600 nm, followed by sampling every 2 h. The OD₆₀₀ values for the original strain at 0 h, 2 h, 4 h, 8 h, 12 h, 18 h, 24 h, 30 h, 36 h, 48 h, 54 h, 60 h and 72 h were determined. The data were recorded and the growth curve was plotted.

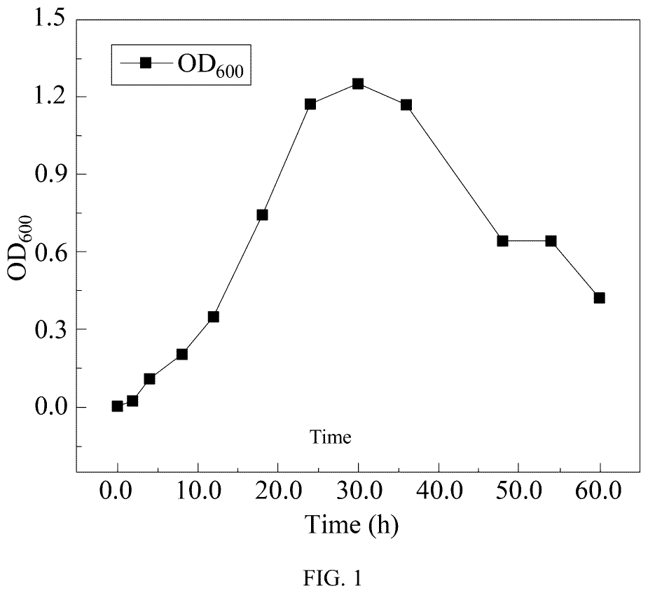

Xanthomonas campestris is a gram-negative pathogen. As an obligate aerobe, it typically exhibits a rod-shaped morphology with a single polar flagellum and demonstrates optimal growth within the temperature range of 25-30° C. The exopolysaccharide secreted by Xanthomonas campestris is referred to as xanthan gum, which is a biogum with superior performance that finds extensive applications across multiple industries and holds enormous market potential. The growth curve of the original strain in liquid medium is as shown in FIG. 1. As can be seen from FIG. 1, the growth curve of the original strain conformed to an “S”-shaped curve, which was consistent with the characteristics of normal microbial growth and reproduction. The period at 0-8 h represented the lag phase of the strain. Within 12-30 h, the strain exhibited exponential growth, with OD₆₀₀ reaching 1.175 at 24 h, which represented the logarithmic growth phase during which the strain exhibited maximal growth rate and peak metabolic activity. Then, growth continued but at a reduced rate, reaching a peak at 30-36 h, which represented the stationary phase of the strain. In subsequent culture, the strain entered the decline phase, during which the GD₆₀₀ decreased slowly and the number of viable bacterial cells in the medium reduced due to bacterial death.

### 4. Heavy Ion Irradiation Mutagenesis

The bacterial solution of the original strain at the logarithmic growth phase was mixed into a uniform bacterial suspension using a vortex mixer. Next, 1 mL of the prepared bacterial suspension was placed in a 35 mm irradiation dish, sealed with a sealing film, and subjected to irradiation mutagenesis using a ¹²C⁶⁺ ion beam generated by the Lanzhou Heavy Ion Research Facility (HIRFL). The ion beam had an extraction energy of 80 MeV/u and an LET of 35.5 keV/mm. A total of 11 mutagenesis doses were selected for irradiation: 0 Gy, 20 Gy, 40 Gy, 60 Gy, 80 Gy, 100 Gy, 120 Gy, 140 Gy, 160 Gy, 180 Gy and 200 Gy.

### 5. Calculation of Lethality Rate

The bacterial solutions with irradiation doses of 0 Gy, 20 Gy, 40 Gy, 60 Gy, 80 Gy, 100 Gy, 120 Gy, 140 Gy, 160 Gy, 180 Gy, and 200 Gy were serially diluted. Then, 40 μL of each of the bacterial suspensions was spread onto a solid medium.

The determination of the optimal dilution gradient is required here. This experiment aims to observe single colonies of Xanthomonas campestris and prevent excessively high bacterial growth density. Culturing the bacteria at the optimal dilution gradient enables cell dispersion, and maintaining the number of colonies on the solid plate to be 50-200 facilitates better observation of bacterial single colonies. As can be seen from Table 1, when the dilution gradient was 10-5, the number of colonies on the plate was less than 200, indicating that the 10-5 gradient is the optimal dilution gradient.

TABLE 1
Dilution gradient

| Dilution gradient | Number of colonies |
|---|---|
| 10-1 | ∞ |
| 10-2 | ∞ |
| 10-3 | ∞ |
| 10-4 | 500+ |
| 10-5 | <200 |
| 10-6 | <30 |

The bacterial suspensions were cultured on a thermostatic shaker at 37° C. and 180 r/min for 96 h, with 3 parallel experiments per group. Then, the number of viable bacteria on the plates at different irradiation doses was recorded. The lethality rate was calculated by dividing the number of colonies after irradiation by the number of colonies in the blank control, and the curve was plotted using the irradiation dose as the abscissa and the lethality rate as the ordinate.

Lethality rate (%)=[1−(number of colonies from irradiation group/number of colonies from control group)]×100%.

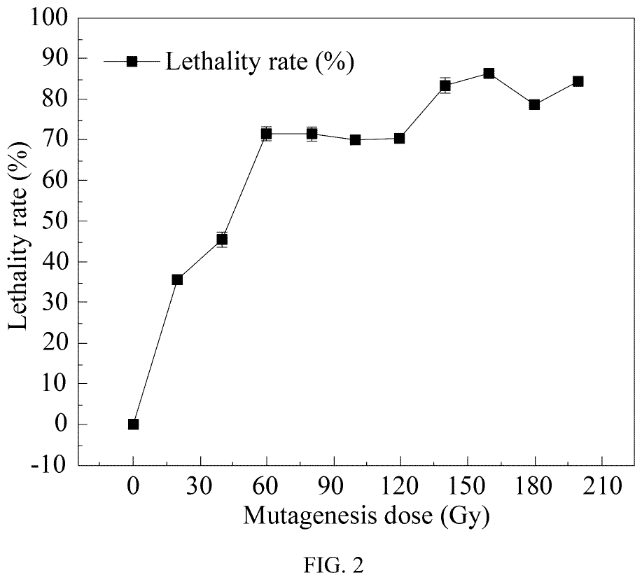

In this test, the heavy ion beam current was controlled within a stable range. Using the relative irradiation dose as the abscissa and the lethality rate as the ordinate, the lethality rate was calculated and the lethality rate curve was plotted. The lethality rates of the original bacterial strain at different irradiation doses are as shown in FIG. 2. As can be seen from FIG. 2, with the increase in irradiation dose, the lethality rate of the original strain generally exhibited a trend of first increasing and then decreasing, presenting a typical “saddle-shaped” pattern. At 160 Gy, the highest lethality rate was found for the strain, at 86.67%. Within the mutagenesis dose range of 0-60 Gy, the bacterial lethality rate increased gradually; within the mutagenesis dose range of 60-120 Gy, the lethality rate plateaus; within the mutagenesis dose range of 120-160 Gy, the lethality rate showed an upward trend; at 180 Gy, the lethality rate decreased slightly; and at 200 Gy, the bacterial lethality rate increased again. When the cells were irradiated, the cell structure was damaged, leading to an increased number of bacterial deaths and a higher lethality rate. Later, bacteria repaired some of the damaged cells, leading to a slight increase in the lethality rate. Therefore, the lethality rate curve showed a trend of increasing, then decreasing, and increasing again.

### 6. Calculation of Positive Mutation Rate

The bacterial suspensions of the original strains after mutagenesis at different irradiation doses were appropriately diluted. Then, 0.1 mL of each of the dilutions was spread onto a solid medium and cultured at 37° C. for 48 h. The morphology and size of colonies at different irradiation doses were observed. Larger colonies were picked up and spot-inoculated onto a screening medium, and then cultured at 37° C. for 72 hours. After colonies grew, Lugol's iodine solution was added dropwise to the screening medium, and clear zones around the colonies were observed. The colony diameter (C) and the clear zone diameter (H) were measured, and the positive mutation rate was calculated according to the formula below. The positive mutation rate curve was plotted using the irradiation time as the abscissa and the positive mutation rate as the ordinate. The strains with larger clear zones around colonies were selected and inoculated into a liquid medium, cultured in a thermostatic shaker at 37° C. and 180 r/min for 24 h, and then stored for the next fermentation screening.

Positive mutation rate (%)=(number of colonies with H/C value of the mutant over 20% higher than that of the original strain/total number of colonies tested)×100%.

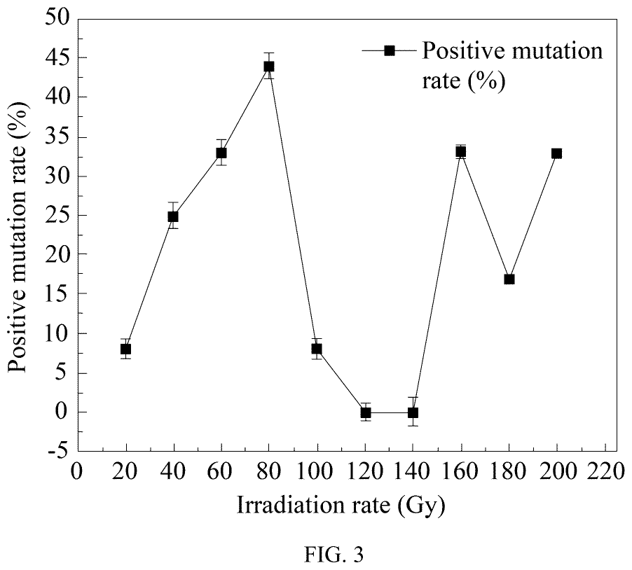

The positive mutation rate of the original strain after mutagenesis was calculated on the basis of the ratio of the clear zone diameter to the colony diameter. Using the relative irradiation dose as the abscissa and the positive mutation rate as the ordinate, the positive mutation rate was calculated and the positive mutation rate curve was plotted. The positive mutation rates of Xanthomonas campestris at different irradiation doses are as shown in FIG. 3. From the observation of FIG. 3, it was seen that with the increase in irradiation dose, the positive mutation rate of the original strain exhibited a trend of increasing, decreasing, increasing, decreasing and then increasing. It can be seen that when the irradiation dose is 80 Gy, the positive mutation rate was 44.00%, reaching the highest, indicating that mutagenesis at an irradiation dose of 80 Gy was favorable for screening for strains with positive mutations. At high irradiation doses, the internal structures of the cells, such as DNA and proteins, are damaged, leading to variations in genetic information. As a result, bacteria are more prone to mutations, making positive mutations more likely to occur. This is favorable for screening for high-yield strains from mutagenized strains.

### 7. Primary Screening of Mutagenized Strains

The morphology and size of colonies at different irradiation doses were observed, and larger, smoother and more translucent colonies were picked and spot-inoculated onto a screening medium, numbered, and then cultured at 37° C. for 72 h. After colonies grew, Lugol's iodine solution was added dropwise to the screening medium, and clear zones around the colonies were observed. The colony diameter (C) and the clear zone diameter (H) were measured, and the H/C value was calculated. The strains with higher H/C ratios were inoculated into a liquid medium and cultured in a theramostatic shaker at 30° C. and 180 r/min for 72 h. These strains were the primarily screened strains.

Sixteen strains with higher H/C ratios were selected from the above steps for culture, and named Y-1, Y-2, Y-3, Y-4, Y-5, Y-6, Y-7, Y-8, Y-9, Y-10, Y-11, Y-12, Y-13, Y-14, Y-15 and Y-16. The 16 primarily screened mutagenized strains were cultured to the logarithmic growth phase, and then inoculated into a fermentation medium for culture. After a certain period of time, xanthan gum was extracted, the gum production rate was determined, and high-yielding strains were selected.

The procedures for the extraction of xanthan gum and the calculation of gum production rate for the strains were as follows:

Extraction of xanthan gum: When the OD₆₀₀ value of the liquid seed was 0.8, the seed solution of the target strain at the logarithmic growth phase was inoculated at a 15% inoculum size into a 250 mL Erlenmeyer flask containing 100 mL of fermentation medium, and then cultured in a thermostatic shaker at 40° C. and 180 r/min for 96 h. Using the ethanol precipitation method, 3 volumes of deionized water were added to the fermentation broth for the dilution of the fermentation broth, and then 3 volumes of absolute ethanol were added for the precipitation of xanthan gum. The mixture was centrifuged at 8000 r/min for 15 min. The supernatant was discarded and the precipitate was retained. The precipitate was then washed twice with absolute ethanol, dried in an oven at 60° C., and ground to obtain the fermentation product xanthan gum, which was weighed.

The fermentation medium composed of 4.5% soluble starch, 0.5% glucose, 1.0% soy protein, 0.1% magnesium sulfate heptahydrate, 0.1% dipotassium hydrogen phosphate and 0.1% potassium dihydrogen phosphate, with a pH of 7.0, was filled at 75 mL/250 mL.

Calculation of gum production rate: The mass of the extracted xanthan gum was divided by the mass of the fermentation broth to obtain the xanthan gum production rate.

$$Xanthan gum production rate ( \% ) = [ mass of xanthan gum sample ( g ) / mass of xanthan gum fermentation broth ( g ) ] \times 100 \% .$$

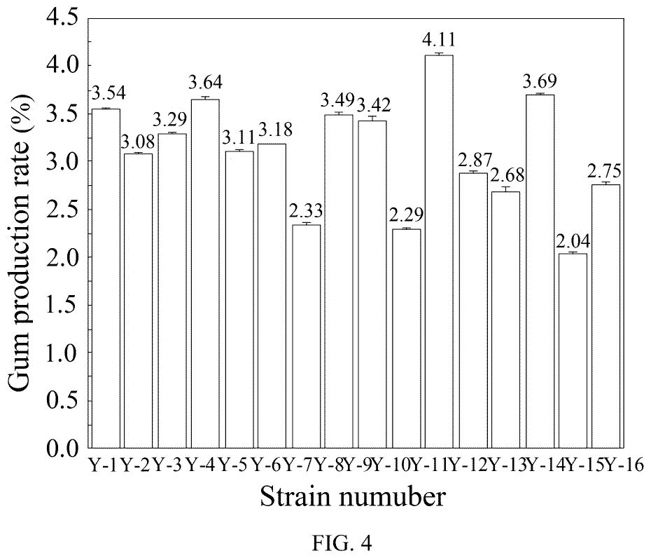

From FIG. 4, it could be observed that six relatively high-yield mutagenized strains Y-1, Y-4, Y-8, Y-9, Y-11 and Y-14 were selected, of which Y-11 demonstrated the highest gum production rate of 4.11%. This indicated that mutagenesis of the original strain using a heavy ion beam could result in xanthan gum-producing strains with higher gum production rates, suggesting the occurrence of positive mutations in the mutagenized strains. The selected six high-yield strains had a strong ability to produce xanthan gum and had better production effects than the original strain. The single colonies of the six mutagenized strains were deposited for the next screening and determination of other properties.

### 8. Secondary Screening of Mutagenized Strains

The primarily selected strains were numbered, cultured to the logarithmic growth phase, inoculated at a 10% inoculum size into 75 mL of fermentation medium, and then cultured in a thermostatic shaker at 37° C. and 180 r/min for 96 h. After the fermentation was completed, the gum production rates of the strains and the light transmittances of aqueous solutions of the fermentation product xanthan gum were determined. The gum production rates and the light transmittances of aqueous solutions of the primarily screened strains were compared. The optimal strains were selected as the secondarily screened strains, and the genetic stability test was performed on the secondarily screened strains.

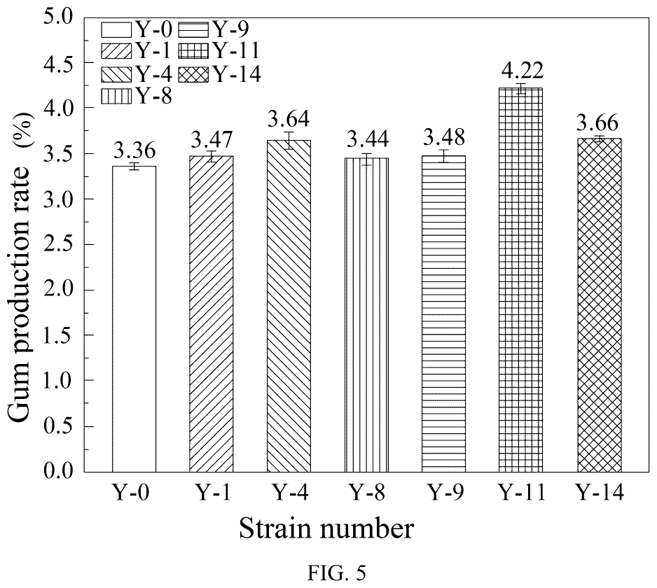

The primarily screened six mutagenized strains in the above steps were inoculated into a fermentation medium, followed by xanthan gum extraction to determine the gum production rates of the strains. As can be seen from FIG. 5, among the six mutagenized strains, Y-11 had a significantly higher gum production rate than the other mutagenized strains, which was 4.22%. Therefore, Y-11 was determined to be the optimal strain relative to the other mutagenized strains.

### 9. Genetic Stability Test

The genetic performance stability test was performed on the selected mutagenized strain. The strain was subjected to 12 consecutive passages under the same culture conditions. The xanthan gum production rate and the light transmittance of aqueous solution of the strain after passage were observed to determine whether or not the strain has genetic stability.

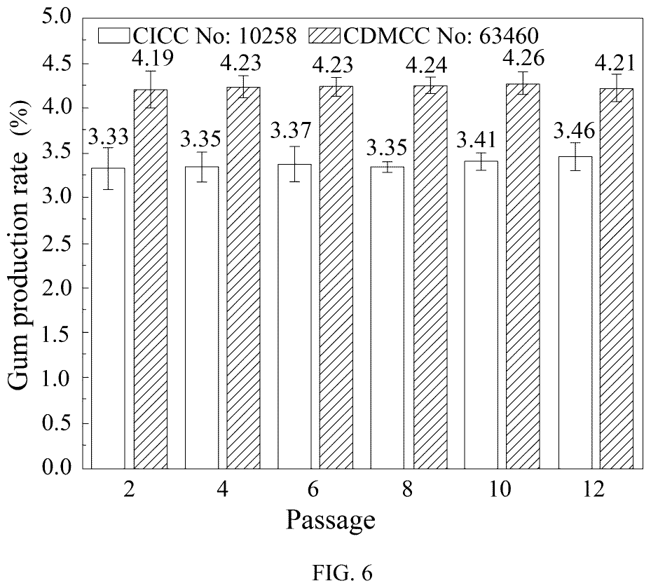

The high-yielding strain Y-11 and the original strain were subjected to 12 consecutive passages under the same conditions and inoculated into a fermentation medium every two generations. The gum production rates of the strains were determined. The results are shown in FIG. 6. The analysis of standard deviation indicated that the gum production rates of the strains showed no significant difference. This indicates that the high-yielding strain Y-11 exhibits stable genetic performance. In this experiment, by using heavy ion mutagenesis, a physical mutagenesis means, for artificial breeding, the Y-11 mutagenized strain with stable performance, low probability of reverse mutation, and high yield can be obtained. The mutagenized strain Y-11 was deposited under a deposit number of GDMCC No: 63460 (hereinafter referred to as “mutagenized strain”), with the taxonomic name: Xanthomonas campestris, deposited with the Guangdong Microbial Culture Collection Center on May 12, 2023.

### 10. Solubility Test

The xanthan gum fermentation products of the selected mutagenized strain and the original strain were each prepared into an aqueous solution at a mass concentration of 1%, which was stirred at 600 r/min until complete dissolution. The time required for complete dissolution of xanthan gum at a mass concentration of 1% was determined. Three parallel experiments were performed for each group, and the data were recorded.

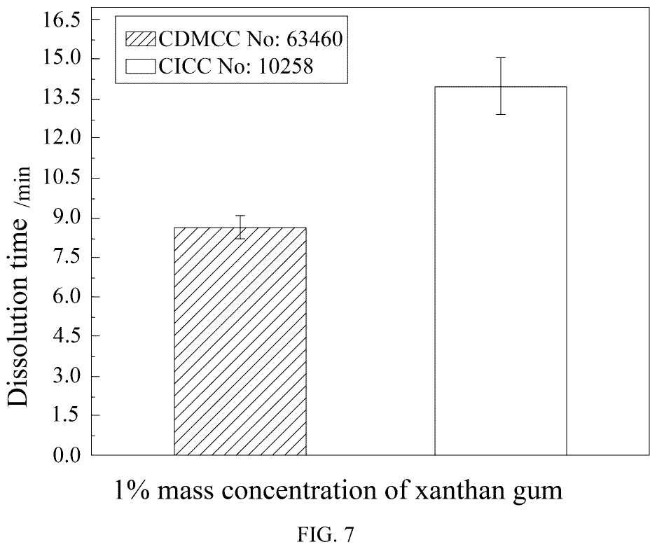

As can be seen from FIG. 7, the complete dissolution time of the xanthan gum fermentation product of the original strain at a mass concentration of 1% was 13.95 min. The xanthan gum fermentation product of the mutagenized strain was prepared into an aqueous solution at a mass concentration of 1%, which was stirred at 600 r/min until complete dissolution. The time required for complete dissolution of xanthan gum at a mass concentration of 1% was determined to be 8.67 min, representing a 60.90% reduction compared to the original strain. This indicates that the xanthan gum fermentation product of the mutagenized strain, after being added at a mass concentration of 1% to a deionized water solution, can completely dissolve within 10 min.

### 11. Temperature Resistance Test

The xanthan gum products obtained by fermentation of the selected mutagenized strain and the original strain were each ground to a powder. 1000 mL of a solution of the xanthan gum product at a mass concentration of 1% was placed in a water bath and heated, with temperatures set at 10° C., 20° C., 30° C., 40° C., 50° C., 60° C., 70° C. and 80° C. The aqueous solution of the xanthan gum product was allowed to stabilize at each temperature for 1 h. An NDJ-5S rotational viscometer was then turned on and set to a rotational speed of 6 r/min, and the viscosity values of the solution of the xanthan gum product at different temperatures were recorded. Three parallel experiments were performed for each group, with units expressed in mPa s.

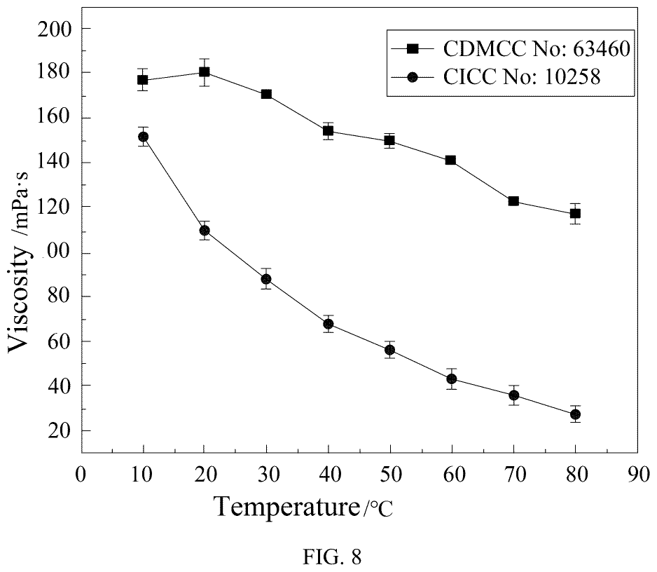

As can be seen from FIG. 8, the viscosity of the aqueous solution of xanthan gum obtained by the fermentation of the mutagenized strain showed a trend of first increasing and then decreasing within the temperature range of 10-80° C. The aqueous solution of xanthan gum exhibited the highest viscosity of 180.34 mPa s at 20° C. The viscosity of the aqueous solution of xanthan gum gradually decreased as the temperature increases, reaching 117.06 mPa s at 80° C. The viscosity showed less significant change throughout this temperature interval, maintaining a moderate level without agglomeration. The viscosity of the aqueous solution of xanthan gum showed no significant change within the temperature range of 10-80° C. compared to the original strain fermentation. The viscosity of the aqueous solution of xanthan gum obtained by the fermentation of the original strain showed a gradually decreasing trend within the temperature range of 10-80° C., reaching 151.43 mPa s at 10° C. When heated to 80° C., the viscosity of the aqueous solution of xanthan gum reaches a minimum of 27.66 mPa s, indicating a relatively significant viscosity change.

Although the examples described above have provided a detailed description of the present disclosure, they are only a part of, rather than all of the embodiments of the present disclosure. All other embodiments that can be obtained according to the examples of the present disclosure without creative efforts shall fall within the scope of protection of the present disclosure.

What is claimed is:

1. (canceled)

2. (canceled)

3. A method for a fermentative preparation of xanthan gum by a Xanthomonas campestris strain Y-11 as a fermentation strain, comprising the following steps:
1) a strain activation: thawing a preserved bacterial solution containing the Xanthomonas campestris strain Y-11 to obtain a thawed bacterial solution, and picking up and streaking the thawed bacterial solution on a plate medium with an inoculating loop; and after a formation of single colonies, picking and inoculating the single colonies at a 1% inoculum size into a first liquid seed medium, and culturing in a thermostatic shaker at a fermentation temperature of 30° C. and 180 r/min for 24 h; and performing two passages to restore an original activity of the Xanthomonas campestris strain Y-11, wherein the Xanthomonas campestris strain Y-11 has a deposit number of GDMCC No: 63460;
2) a liquid seed culture: inoculating an activated Xanthomonas campestris strain Y-11 at the 1% inoculum size into a second liquid seed medium, and culturing in the thermostatic shaker at the fermentation temperature of 30° C. and 180 r/min for 24 h to obtain liquid seeds at a logarithmic growth phase to complete the liquid seed culture;
3) a fermentation culture: inoculating the liquid seeds at the logarithmic growth phase at a 5-20% inoculum size into a fermentation medium, and culturing in the thermostatic shaker at a fermentation temperature of 30-40° C. and 180 r/min for 96 h; and
4) a xanthan gum extraction: extracting the xanthan gum from a fermentation broth by an ethanol precipitation method.

4. The method according to claim 3, wherein the plate medium in the step 1) comprises the following components by weight percentage: 0.5% soluble starch, 1% peptone, 0.3% beef extract, 0.5% sodium chloride, and 2% agar, with a pH value of 6.5-7.0, the plate medium is sterilized at 115° C. for 30 min, and a culture time on the plate medium is 72 h.

5. The method according to claim 3, wherein the first liquid seed medium in the step 1) and the second liquid seed medium in the step 2) both-comprise the following components by weight percentage: 2.0% soluble starch, 0.5% peptone, 0.3% potassium dihydrogen phosphate, and 0.2% sodium chloride, with a pH value of 7.0.

6. The method according to claim 3, wherein an inoculation in the step 3) is performed at an inoculum size of 15%.

7. The method according to claim 3, wherein the fermentation culture in the step 3) is performed at 40° C.

8. The method according to claim 3, wherein the fermentation medium in the step 3) comprises the following components by weight percentage: 4.5% soluble starch, 0.5% glucose, 1.0% soy protein, 0.1% magnesium sulfate heptahydrate, 0.1% dipotassium hydrogen phosphate, and 0.1% potassium dihydrogen phosphate, with a pH value of 7.0.

9. The method according to claim 3, wherein the extracting the xanthan gum from the fermentation broth by the ethanol precipitation method in the step 4) comprises: mixing the fermentation broth with absolute ethanol to obtain a mixture, and centrifuging the mixture to obtain the xanthan gum.

10. The method according to claim 9, wherein before mixing with the absolute ethanol, the fermentation broth is diluted by mixing with deionized water to obtain a diluted solution, with a volume ratio of the diluted solution to the deionized water being 1:3.
